(19) United States
(12) Patent Application Publication
(10) Pub. No.: US 2006/0293282 A1
(43) Pub. Date: Dec. 28, 2006

(54) Substituted aminoalkanephosphonic acids

(76) Inventors: Kurt Lingenhohl, WEIL AM RHEIM (DE); Yves Auberson, Allschwil (CH); Alyson Fox, London (GB); Hans Ch. Neijt, Himmelried (CH); Hans O. Kalkman, Basel (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/512,923

(22) PCT Filed: Apr. 29, 2003
(86) PCT No.: PCT/EP03/04466

§ 371 Date: Oct. 29, 2004

(30) Foreign Application Priority Data
Apr. 30, 2002 (GB) 0209886.1
Apr. 30, 2002 (GB) 0209887.9
Apr. 30, 2002 (GB) 0209889.5
May 7, 2002 (GB) 0210371.1
May 31, 2002 (GB) 0212760.3

(51) Int. Cl. A61K 31/675

(52) U.S. Cl. 514/80

(57) ABSTRACT

The present invention relates the use of substituted aminoalkanephosphonic acids in treating neuropathic pain, affective and attention disorders, schizophrenia, tinnitus, myopia and other ocular disorders.

The present invention relates to new pharmaceutical uses of substituted aminoalkanephosphonic acids.

More particularly the invention relates to new pharmaceutical uses for compounds of formula I 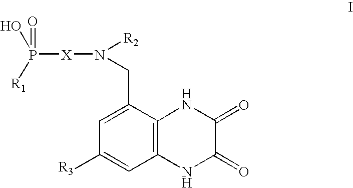
wherein
R₁ is hydroxy or (C₁₋₄)alkyl,
R₂ is (C₁₋₄)alkyl,
R₃ is hydrogen, (C₁₋₄) alkyl, fluorine, chlorine, bromine, trifluoromethyl, cyano or nitro, and
X is (C₁₋₆)alkylene, (C₁₋₄)alkylidene, (C₁₋₆)alkylene(C₃₋₆)cycloalkylene or (C₁₋₆)alkylene-(C₃₋₆)cycloalkylidene,
   and their pharmaceutically acceptable salts, hereafter referred to as “the compounds”.

The compounds as well as their production process are known e.g. from WO 98/17672.

This application also discloses the use of the compounds for the treatment of pathological conditions which respond to blockade of excitatory amino acid receptors, such as AMPA receptors, NMDA, kainate receptors and glycine binding sites of NMDA receptors, for example of neurodegenerative disorders, stroke, epilepsy, anxiety and pain.

In accordance with the present invention, it has now surprisingly been found that the compounds are also useful in the treatment of neuropathic pain.

The activity of the compounds in the treatment of neuropathic pain is evidenced, for example, in the following model of neuropathic pain in the rat:

Wistar rats are anaesthetised with enflurane and a small incision is made mid-way up one thigh to expose the sciatic nerve. The nerve is cleared of connective tissue and a 7-0 silk suture is inserted into the nerve using a ⅜ curved reverse-cutting min-needle, and tightly ligated so that the dorsal ⅓ to ½ of the nerve thickness is held within the ligature. The muscle and skin are closed with sutures and clips and the wound dusted with antibiotic powder. This procedure produces a mechanical hyperalgesia which develops within 2-3 days and is maintained for at least 4 weeks. Mechanical hyperalgesia is assessed by measuring paw withdrawal thresholds on both the ipsilateral (ligated) and contralateral (unligated) hindpaw to an increasing pressure stimulus applied to the paw using an analgesymeter (Ugo-Basile) with a wedge-shaped probe (area 1.75 mm²) and a cut-off threshold of 250 g. The end point is taken as the first sign of pain response (struggling, vocalisation or paw withdrawal). Hyperalgesia is indicated by the difference in ipsilateral and contralateral withdrawal thresholds. Reversal of established hyperalgesia by administered compounds is measured 12-14 days following surgery, using 6 animals per treatment group. Paw withdrawal thresholds are measured prior to and then up to 6 hours following drug or vehicle administration. Statistical analysis is carried out on withdrawal threshold readings using ANOVA followed by Tukey's HSD test comparing drug treated and time-matched vehicle treated animals.

In this model, the compounds significantly reverse neuropathic mechanical hyperalgesia at 10 mg/kg p.o. With the compound {[(7-nitro-2,3-dioxo-1,2,3,4-tetrahydro-quinoxalin-5-ylmethyl)-amino]-methyl}-phosphonic acid, for example, a maximal reversal of neuropathic mechanical hyperalgesia of 35% is achieved after 3 hours on adminstration of 10 mg/kg p.o.

The activity of the compounds of formula I in the treatment of neuropathic pain can be confirmed in clinical trials, for example in the following study aimed at evaluating the efficacy of a compound in treating chronic pain in patients with diabetic neuropathy:

Patients are randomized to receive 2400 mg/day of the compound or placebo in a 1:1 ratio.

The study consists of a Pre-randomization Phase (1 week) and a Double-blind Phase (5 weeks). The double-blind Phase consists of three periods: a one week Titration Period, a three-week Maintenance Period and a one-week Follow-up Period.

During the 1-week Pre-randomization Phase, the eligibility of the patients is evaluated. Patents meeting all inclusion/exclusion criteria are randomized to either the compound or placebo in the Double-blind Phase. During the 1-week Titration Period, study medication is up-titrated from 800 mg/day (given b.i.d.) to 2400 mg/day (given b.i.d.). Patients who complete the 1-week Titration Period then enter the 3-week Maintenance Period. Patients who complete the 3-week Maintenance period or prematurely discontinue double-blind treatment then enter the 1-week Follow-up Period. Study medication is completely withdrawn on entry into the Follow-up Period. During the Double-blind Phase, serial efficacy and safety assessments are obtained.

120 male and female outpatients, aged 18-65 years with a clinical diagnosis of diabetes mellitus (type I or II) and a history of pain associated with diabetic neuropathy for 6 months to 3 years prior to study entry, are randomized 1:1 to the compound or placebo.

The total score of the Short-Form McGill Pain Questionnaire (SF-MPQ) at the end of Maintenence Period is used as primary efficacy parameter. Average weekly pain severity rating (daily patient pain diary) from start of randomized treatment to end of Maintenance Period, usage of rescue medication during the Titration and Maintenance Period, and average pain severity rating during the Follow-up Period (rebound pain), are used as secondary efficacy parameters.

The SF-MPQ total pain score at the end of the Maintenance Period is analyzed using an analysis of covariance model adjusting for the effect of treatment on post-treatment scores by using the baseline SF-MPQ total pain score as a covariate. Average weekly pain severity is analyzed using an analysis of covariance model with repeated measures using the treatment week and the mean pain severity rating during the Pre-randomization Phase as covariates. Usage of rescue medication during the Double-blind Phase is analyzed using the Cochran-Mantel-Haenszel test controlling for center. The mean pain severity rating during the Follow-up Period (rebound pain) is analyzed using an analysis of covariance model adjusting for the effect of treatment on the mean pain severity rating of the Follow-up Period with the mean pain severity rating during the Prerandomization Phase as a covariate.

In this study, the compounds, more particularly {[(7-nitro-2,3-dioxo-1,2,3,4-tetrahydro-quinoxalin-5-ylmethyl)-amino]-methyl}phosphonic acid are found to decrease pain severity ratings relative to placebo during the Maintenance and Follow-up Periods, in a statistically. significant way.

The compounds are therefore useful in the treatment of neuropathic pain and associated hyperalgesia, including trigeminal and herpetic neuralgia, diabetic neuropathic pain, migraine, causalgia and differentiation syndromes such as brachial plexus avulsion.

In a further aspect of the present invention, it has surprisingly been found that the compounds are also useful in the treatment of affective and attention disorders.

The activity of the compounds in the treatment of affective disorders including bipolar disorders, e.g. manic-depressive psychoses, extreme psychotic states e.g. mania, is evidenced, for example, in the following tests suitable for detecting drugs reversing psycho-motor stimulatory effects.

Test 1: NMDA-Antagonist Induced Locomotion:

Male Wistar Kyoto rats (Iffa Crédo, Lyon, France) weighing between 250 and 310 g are used. In principle 4 treatment groups are formed: 1) the compound (doses 1, 3 or 10 mg/kg) followed by the competitive NMDA receptor antagonist (S)-2-amino-3-(2′-chloro-5-phosphonomethyl-biphenyl-3-yl)-propionic acid, hereinafter SDZ 220-581 (10 mg/kg), 2) solvent-pretreatment followed by SDZ 220-581 (10 mg/kg), 3) solvent followed by solvent, 4) the compound (1, 3, 10 mg/kg) followed by solvent. Rats are randomly allocated to these pretreatment groups (n=10/dose group). Drugs are administered subcutaneous (s.c.), 15 min prior to SDZ 220-581. Immediately after the animals received SDZ 220-581, they are placed into the activity monitor for a period of 60 min. Locomotor activity is analysed over the initial 30 minutes.

Locomotion is recorded with a videotracking system (VideoMot2, TSE Technical and Scientific Equipment GmbH, Bad Hombourg, Germany), using a closed circuit digital videocamera (WV-BP.330/GE, Panasonic, Osaka, Japan). The video-signal from the camera is digitized and used for data analysis. Animals are on a normal 12/12 h day-night cycle, with light on at 06:00H. Experiments are performed in a dimly lit room between 07:00 H and 15:00H. Animals are placed in a round arena (diameter 42 cm, height 32 cm) made of grey polyvinylchloride plastic. The camera is placed such, that four animals (one per arena) can be recorded simultaneously.

In this test, the compounds (1-10 mg/kg, s.c.) do not significantly alter locomotor activity as compared to vehicle-treated animals at any time during a period of 30 min. However, the competitive NMDA receptor antagonist SDZ 220-581 (10 mg/kg, s.c.) induces a strong locomotor response. Thus, whereas control animals walk approximately 8-10 m during 30 min, SDZ 220-581-treated animals walked approximately 30 m. This locomotor response is reduced in a dose dependent manner by the compounds. With {[(7-nitro-2,3-dioxo-1,2,3,4-tetrahydro-quinoxalin-5-ylmethyl)-amino]methyl}-phosphonic acid (10 mg/kg), the effect of the NMDA-antagonist SDZ 220-581 is almost normalized.

Test 2: NMDA-Channel Blocker Induced Head Swaying and Circling:

Adult male Wistar Kyoto rats (340-380 g; Iffa Credo, Lyon, France) are used. The animals are randomized to the following treatment groups (n=10 per group): the compound (dosed 0, 3 or 10 mg/kg) followed by phencyclidine (PCP; an NMDA channel blocker, dosed 0 or 10 mg/kg). Compound (at t=−15 min) and PCP (at t=0 min) are administered s.c. in a volume of 1 ml/kg. Video-recordings of the animals behaviour over the period 0-30 min following PCP are scored by an observer who is unaware about the animals pretreatment. Head-swaying (rocking the head repeatedly by at least 2 cm left and right) and circling (turning around by using the forepaws, whereas the hindpaws remain more or less on the original position) are scored as present (1) or absent (0), every five minutes for the duruation of 1 minute. The scores for individual animals is summed and group scores used for statistical analysis (t-test with Bonferroni correction).

In this test, PCP (10 mg/kg, s.c.) induces weak head-swaying and circling. Pretreatment with the compounds (3 and 10 mg/kg, s.c.) significantly enhances these behavioural responses to PCP(P<0.05).

NMDA-antagonist induced locomotor responses reflect a mania-like state. Blockade of this activity indicates an anti-manic activity. Furthermore, enhancement of head-swaying and circling suggest a behavioural desinhibition (=anxiolytic-/antidepressant-like) and sociotropic activity. Therefore, the compounds are useful in the treatment of affective disorders including bipolar disorders, e.g. manic-depressive psychoses, extreme psychotic states e.g. mania and excessive mood swings where behavioural stabilization is desired. In addition, the compounds are indicated in ADHD (attention deficit hyperactivity disorders) and other attention disorders, e.g. autism, anxiety states, generalized anxiety and agoraphobia, as well as those behavioural states characterized by social withdrawal e.g. negative symptoms.

In a further aspect of the present invention, it has surprisingly been found that the compounds are also useful in the treatment of schizophrenia and psychosis like symptoms in other indications, e.g. Parkinson's disease.

The antischizophrenic activity of the compounds is indicated in standard tests, e.g. in the amphetamine-induced hyperlocomotion test. Blockade of amphetamine-induced hyperlocomotion is well known as screening paradigm for antischizophrenic activity.

Male Wistar rats (Iffa Crédo, Lyon, France) weighing between 215 and 315 g are used. In principle 4 treatment groups are formed: 1) the compound (doses 1, 3 or 10 mg/kg) followed. by amphetamine (1 mg/kg), 2) solvent-pretreatment followed by amphetamine (1 mg/kg), 3) solvent followed by solvent, 4) the compound (10 mg/kg) followed by solvent. Rats are randomly allocated to these pretreatment groups (n=10/dose group). Drugs are administered subcutaneous (s.c.), 15 min prior to amphetamine. Immediately after the animals received amphetamine, they are placed into the activity monitor for a period of 60 min. Locomotor activity is analysed over the initial 30 minutes.

Locomotion is recorded with a videotracking system (VideoMot2, TSE Technical and Scientific Equipment GmbH, Bad Hombourg, Germany), using a closed circuit digital videocamera (WV-BP.330/GE, Panasonic, Osaka, Japan). The video-signal from the camera is digitized and used for data analysis. Animals are on a normal 12/12 h. day-night cycle, with light on at 06:00H. Experiments are performed in a dimly lit room between 07:00H and 15:00H. Animals are placed in a round arena (diameter 42 cm, height 32 cm) made of grey polyvinylchloride plastic. The camera is placed such, that four animals (one per arena) can be recorded simultaneously.

Amphetamine is dissolved in physiological saline as 1 mg/ml and administered s.c. In a volume of 1 ml/kg. The compound is dissolved in a few drops of NaOH (0.1 N) and further diluted with physiological saline as required to obtain solutions of 10, 3 and 1 mg/ml. It is administered s.c. in a volume of 1 ml/kg.

Comparison between groups is done with Student's t-test, corrected for multiple testing using the Bonferroni procedure.

In this test, the compounds reduce the amphetamine-induced locomotion at doses of about 1 mg to about 10 mg/kg s.c.

In still a further aspect of the present invention, it has surprisingly been found that the compounds are also useful in the treatment of tinnitus.

The activity in tinnitus of the compounds is indicated in standard tests, e.g. in the salicylate-induced tinnitus model.

It has been demonstrated [C. A. Bauer et al., Hearing Research 147 (2000) 175-182] that chronic salicylate exposure causes upregulation of glutamic acid decarboxylase (GAD) expression in the rat inferior collicuius (IC), associated with the development of tinnitus. Furthermore, electrophysiological recordings from auditory neurons using patch clamp recording techniques [D. Peruzzi et al. Neuroscience 101 (2000) 403-416, X. Lin et al., Journal of Neurophysiology 79 (1998) 2503-2512] and single neuron recordings [J. J. Eggermont and M. Kenmochi, Hearing Research 117 (1998) 149-160] showed that the excitability of neurons is changed following salicylate and quinine treatment.

Administration of salicylate or quinine caused an increase in the firing rate auditory neurons measured by extracellular electrophysiological recording techniques. Using in vitro electrophysiological recording techniques superfusion with salicylate increases the excitability of the recorded neurons. On administration of the compounds at concentrations of about 1 nM to 100 μM, the effects of salicylate were reversed.

For the treatment of the above mentioned indications, appropriate dosage will of course vary depending upon, for example, the compound employed, the host, the mode of administration and the nature and severity of the condition being treated. However, in general, satisfactory results in animals are indicated to be obtained at a daily dosage of from about 1 to about 50 mg/kg animal body weight in larger mammals, for example humans, an indicated daily dosage is in the range from about 10 to about 1000 mg of a compound according to the invention, conveniently administered, for example, in divided doses up to four times a day.

In a further aspect of the present invention, it has surprisingly been found that the compounds are also useful in the treatment of myopia and other ocular disorders.

Such disorders include, but are not limited to, age-related macular degeneration, diabetic retinopathy, cystoid macular edema (CME), pathologic myopia, Leber's hereditary optic neuropathy, retinitis pigmentosa, and other hereditary retinal degenerations.

The activity against myopia of the compounds is indicated in standard tests, e.g. in the model according to R. A. Stone et al. [Proc. Natl. Acad. Sci. (USA) 86, 704-706 (1989)] wherein experimental myopia is produced in chicken, on administration of about 0.1 to about 1 mg/kg in eye drops.

Efficacy in the described ocular disorders might be established for example in the following animal models:

1) Spontaneous development of a secondary form of glaucoma (e.g. pigment dispersion, angle closure or angle dysgenesis) in mice (for example, but not exclusively, strains DBA/2J, DBA/2Nnia, and AKXD281/Ty mice as described in Anderson et al., BMC Genetics 2001; 2:1, Chang et al., Nature Genetics 1999; 21: 405-409, John et al., Invest. Ophthalmol. Vis. Sci. 1998; 39: 951-962, Sheldon et al., Lab. Animal Sci. 1995; 15:508-518)

2) Genetic animal models for retinal degeneration, e.g. rd mouse (as described in Li et al., Invest. Ophthalmol. Vis. Sci. 2001; 42: 2981-2989), Rpe65-deficient mouse (Van Hooser et al., PNAS 2000.; 97: 8623-8628), RCS rat (Faktorovich et al., Nature 1990; 347:83-86), rds mouse (Ali et al., Nature Genetics 2000, 25: 306-310), rcd1 dog (Suber et al., PNAS 1993; 90: 3968-3972)

3) Experimental retinal degeneration induced by

light exposure in mice (as described in Wenzel et al., Invest. Ophthalmol. Vis. Sci. 2001; 42: 1653-1659) or rats (Faktorovich et al., J. Neurosci: 1992; 12: 3554-3567)

administration of N-methyl-N-nitrosourea (Kiuchi et al., Exp. Eye Res. 2002; 74:383-392) or sodium iodate (Sorsby & Harding, Vision Res. 1962; 2: 139-148).

4) Experimental model for the injury of the optic nerve (ON)

by ON crush in mice (Levkovitch-Verbin et al., Invest Ophthalmol. Vis. Sci. 2000; 41: 4169-4174) and rats (Yoles and Schwartz, Exp. Neurol. 1998; 153:1-7)

by ON transection in rats (as described in Martin et al., Invest. Ophthalmol. Vis. Sci. 2002; 43: 2236-2243, Solomon et al. J. Neurosci. Methods 1996; 70:21-25)

by experimental transient (acute) retinal ischemia in rats after ophthalmic vessel ligature (as described in Lafuente et al., Invest. Ophthalmol. Vis. Sci. 2001; 42:2074-2084) or cannulation of the anterior chamber (Buchi et al., Ophthalmologic 1991; 203:138-147)

by intraocular endothelin-1 injection in rats (Stokely at al., Invest. Ophthalmol. Vis. Sci. 2002; 43: 3223-3230) or rabbits (Takei et al., Graefes Arch. Clin. Exp. Ophthalmol 1993; 231:476-481).

For the treatment of myopia and other ocular disorders, appropriate dosage will of course vary depending upon, for example, the compound employed, the host, the mode of administration and the nature and severity of the myopia. However, in general, satisfactory results in animals are indicated to be obtained at a daily dosage of from about 0.01 to about 1 mg/kg animal body weight. In larger mammals, for example humans, an indicated daily dosage is in the range from about 0.25 to about 10 mg of a compound according to the invention, conveniently administered, for example, in divided doses up to four times a day.

For the above mentioned indications, the compounds may be administered in any usual manner, e.g. orally, for example in the form of tablets or capsules, or parenterally, for example in the form of injection solutions or suspensions.

For the treatment of myopia and other ocular disorders, the compounds may be administered topically in or around the eye, for example as eyedrops, ophthalmic suspensions or ointments, subconjunctival, peribulbar, retrobulbar or intravitreal injections, possibly with the use of slow-release devices, such as conjunctival inserts, microspheres or other periocular or intraocular depot devices.

The compounds are preferably applied topically to the eye in ca. 0.002 to ca. 0.02% ophthalmological solutions. The ophthalmic vehicle is such that the compound is maintained in contact with the ocular surface for a sufficient time period to allow the compound to penetrate the corneal and internal regions of the eye. The pharmaceutically acceptable ophthalmic vehicle may be e.g. and ointment, vegetable oil, or encapsulating material.

Suitable compounds for the treatment of the above mentioned indications include {[(7-nitro-2,3-dioxo-1,2,3,4-tetrahydro-quinoxaline-5-ylmethyl)-amino]-methyl}-phosphonic acid, (R)-N-(2,3-dioxo-7-nitro-1,2,3,4-tetrahydroquinoxaline-5-ylmethyl)-α-(ethylamino)-ethylphosphonic acid, (S)-N-(7-bromo-2,3-dioxo-1,2,3,4-tetrahydroquinoxaline-5-ylmethyl)-α-aminoethylphosphonic acid and their pharmaceutically acceptable salts.

The present invention also provides pharmaceutical compositions comprising a compound of formula I in association with at least one pharmaceutical carrier or diluent, for use in the treatment of neuropathic pain, affective and attention disorders, schizophrenia, tinnitus, myopia and other ocular disorders. Such compositions may be manufactured in conventional manner. Unit dosage forms for the treatment of neuropathic pain, affective and attention disorders, schizophrenia and tinnitus may contain for example from about 2.5 mg to about 500 mg of the compound of formula I. Unit dosage forms for the treatment of myopia and other ocular disorders may contain for example from about 0.05 mg to about 5 mg of the compound of formula I.

The invention further provides the use of a compound of formula I for the manufacture of a pharmaceutical composition for the treatment of neuropathic pain, affective and attention disorders, schizophrenia, tinnitus, myopia and other ocular disorders.

The invention furthermore provides a method for the treatment of neuropathic pain, affective and attention disorders, schizophrenia, tinnitus, myopia and other ocular disorders in a subject in need of such treatment, which comprises administering to said subject a therapeutically effective amount of a compound of formula I.

1. A method of treatment for neuropathic pain, affective and attention disorders, schizophrenia, tinnitus, myopia and other ocular disorders in a subject in need of such treatment. which comprises administering to said subject a therapeutically effective amount of a compound of formula I: 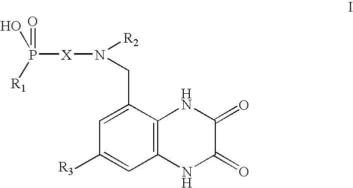
wherein
R₁ is hydroxy or (C₁₋₄)alkyl,
R₂ is (C₁₋₄)alkyl
R₃ is hydrogen, (C₁₋₄) alkyl, fluorine, chlorine, bromine, trifluoromethyl, cyano or nitro, and
X is (C₁₋₆)alkylene, (C₁₋₆)alkylidene, (C₁₋₆)alkylene(C₃₋₆)cycloalkylene or (C₁₋₆)alkylene(C₃₋₆)cycloalkylidene,
in free or in pharmaceutically acceptable salt form.

2. The method of treatment of claim 1, wherein the compound of formula I is {[(7-nitro-2,3-dioxo-1,2,3,4-tetrahydro-quinoxaline-5-ylmethyl)-amino]-methyl}-phosphonic acid, (R)-N-(2,3-dioxo-7-nitro-1,2,3,4-tetrahydroquinoxaline-5-ylmethyl)-α-(ethylamino)-ethylphosphonic acid or (S)-N-(7-bromo-2,3-dioxo-1,2,3,4-tetrahydroquinoxaline-5-ylmethyl)-α-aminoethylphosphonic acid, in free or pharmaceutically acceptable salt form.

3. A pharmaceutical composition which incorporates as active agent a compound of formula I according to claim 1 in free or pharmaceutically acceptable salt form, for use in the treatment of neuropathic pain, affective and attention disorders, schizophrenia, tinnitus, myopia and other ocular disorders.

4. (canceled)

5. (canceled)
